(19) United States
(12) Patent Application Publication
(10) Pub. No.: US 2022/0296095 A1
(43) Pub. Date: Sep. 22, 2022

(54) APPARATUS FOR OCT-BASED IMAGING, MICROSCOPY SYSTEM AND METHOD FOR OPERATING A MICROSCOPY SYSTEM

(71) Applicant: Carl Zeiss Meditec AG, Jena (DE)

(72) Inventors: Hessam Roodaki, Muenchen (DE); Abouzar Eslami, Muenchen (DE)

(21) Appl. No.: 17/699,045

(22) Filed: Mar. 18, 2022

(30) Foreign Application Priority Data
Mar. 18, 2021 (DE) 10 2021 202 626.5

(51) Int. Cl. A61B 3/10; G02B 21/00; A61B 3/13; A61B 3/00; A61B 3/14

(52) U.S. Cl. CPC A61B 3/102; G02B 21/0032; G02B 21/008; G02B 21/0056; G02B 21/0036; G02B 21/0028; A61B 3/13; A61B 3/0066; A61B 3/14

(57) ABSTRACT

An apparatus for OCT-based imaging, a microscopy system and a method for operating a microscopy system, include at least one OCT radiation source and at least one connection device for connecting the apparatus to the microscopy system, an optical connection configured to be established between the OCT radiation source and the light-guide element of the apparatus in a connected state, wherein the microscopy system includes means for detecting a connection state change, and a change-conditional adjustment of the mode of operation of the microscopy system being performable upon the detection of a connection state change.

### CROSS REFERENCE TO RELATED APPLICATIONS

This application claims priority to German patent application DE 10 2021 202 626.5, filed Mar. 18, 2021, the entire content of which is incorporated herein by reference.

### TECHNICAL FIELD

The disclosure relates to an apparatus for optical coherence tomography (OCT)-based imaging, a microscopy system and to a method for operating a microscopy system.

### BACKGROUND

OCT denotes an imaging method for generating one-dimensional, two-dimensional or even three-dimensional image recordings of scattering materials, for example biological materials, with a micrometer resolution. For OCT imaging, light is radiated at an examination object and the light reflected by the examination object is superimposed with light in a reference path, an image information item being obtained by evaluating the interference pattern of this superposition.

Imaging in medical applications, for example in the field of ophthalmology, is one field of use for OCT. Further medical applications include cancer diagnostics and skin examinations. In ophthalmology, OCT imaging is used, inter alia, to examine the fundus, in particular the retina, for example for diagnosing diseases such as macular degeneration, and also other diseases.

The integration of an OCT system in a surgical microscope is also known. Thus, US 2020/0129067 A1 describes an OCT system including an OCT light source and an OCT evaluation unit, a first OCT light guide being guided to a surgical microscope and a second OCT light guide being guided to a surgical instrument. A switching module, through which the light from the OCT light source is guided to the surgical microscope or to the surgical instrument, has also been described.

US 2017/0280989 A1 is also known, which describes an ophthalmological visualization system. In particular, a surgical microscope with an integrated OCT system is described. Further described is an instrument for OCT imaging which may be introduced into the patient's eye for example. In this context, the surgical microscope is described as being able to include a source selection unit, with which it is possible to choose either OCT images generated by an OCT system of the microscope or OCT images generated by an OCT system of the instrument.

DE 10 2015 116 215 B3 is also known; it describes a visualization system for eye surgery. This visualization system includes a surgical microscope with an optical coherence tomography device for generating OCT image data. A probe that is insertable into the eye and has a second optical coherence tomography device for generating second OCT image data of structures of the eye is also disclosed. The document further describes an optical switch which allows an OCT base unit to be connected to an optical fiber that leads to the aforementioned probe. Alternatively, the optical switch may connect the optical base unit to a scanner in order to bring about OCT volume imaging of the eye by way of the microscope.

The aforementioned documents do not disclose that the probe connected to the OCT unit of a microscope is interchangeable. However, it is desirable to use different apparatuses or instruments for OCT-based imaging and consequently connect these to a microscopy system with an OCT radiation source, for example in order to facilitate various medical applications.

However, a problem arising is that when different external apparatuses for OCT imaging are connected to such a microscopy system it is necessary to set a mode of operation of the microscopy system, in particular of the OCT unit, that is adapted to the apparatus in order to facilitate OCT imaging of the desired quality using the connected apparatus. When detaching a connected apparatus, it may also be necessary to readjust the mode of operation of the microscopy system. This adjustment of the mode of operation, which is generally performed by hand, is time consuming and not very convenient for the user.

Thus, a technical problem arising is that of developing an apparatus for OCT-based imaging, a microscopy system and a method for operating a microscopy system, which ensure a quick adaptation of the mode of operation of the microscopy system when an external apparatus for OCT-based imaging is connected to or detached from the microscopy system and ensure great user-friendliness.

### SUMMARY

The technical problem is resolved by providing an apparatus for OCT-based imaging, a microscopy system, and a method for operating a microscopy system as described herein.

An apparatus for OCT-based imaging can be, e.g., an imaging probe, for example a probe that is insertable into an examination region, for example an eye, which probe serves to generate OCT image representations of the said examination region. However, it is also conceivable for the apparatus to be a medical instrument, for example forceps, a suction apparatus, a scalpel, or any other medical instrument, which additionally facilitates OCT imaging.

The apparatus includes at least one light-guide element for guiding the radiation required for OCT-based imaging, that is to say in particular for guiding the radiation generated by an OCT radiation source and the radiation reflected by the examination region. By way of example, this light-guide element can be in the form of a light waveguide or optical fiber.

Further, the apparatus includes at least one connector device for connecting the apparatus to a microscopy system with an OCT radiation source. In particular, the OCT radiation source can be a laser light source. In this case, the wavelength and/or the power of the radiation generated by the OCT radiation source may be adjustable.

In addition to the OCT radiation source, the microscopy system may also include an OCT evaluation device for generating the image information items and an OCT reference beam path. The OCT evaluation device may be configured such that it is possible to carry out optical coherence tomography according to what is known as the SD-OCT, TD-OCT or SS-OCT principle. The OCT evaluation device may include an OCT detector or be formed as such. The OCT radiation source, the OCT evaluation device and optionally the OCT reference beam path as well may be part of an OCT unit of the microscopy system.

Further, the microscopy system may include at least one first microscopy system-side light-guide element for guiding the radiation generated by the OCT radiation source. This light-guide element may connect the radiation source to a beam path of the microscopy system. Hence, the OCT radiation from the microscopy system-side light-guide element may be coupled into the beam path of the microscopy system and radiated at an examination region in a manner known per se, for example by way of an optical element of the microscopy system. Further, light from the beam path of the microscopy system may be coupled into the microscopy system-side light-guide element and guided to the OCT evaluation device.

Further, the microscopy system may include a further microscopy system-side light-guide element which optically connects a (microscopy system-side) connection device for the apparatus to the OCT radiation source and/or optically connects this connection device to the OCT evaluation device. It is possible that this further microscopy system-side light-guide element is connected to a portion of the above-described first microscopy system-side light-guide element.

Further, the microscopy system may include an optical switch which, in a first switching state, connects the OCT radiation source to the beam path of the microscopy system and/or the beam path of the microscopy system to the OCT evaluation device and, in a further switching state, connects the OCT radiation source to the microscopy system-side connection device and/or the microscopy system-side connection device to the OCT evaluation device.

An optical connection between the OCT radiation source and the light-guide element of the apparatus is establishable or established in a connected state, that is to say in a state in which the connection device of the apparatus, which may also be referred to as apparatus-side connection device, is connected to the microscopy system. An optical connection between the apparatus-side light-guide element and the above-described OCT evaluation device may likewise be establishable.

The connection device can consequently serve to establish an optical connection between the OCT radiation source and the apparatus-side light-guide element. Additionally, the connection device may serve to establish a mechanical connection between the apparatus and the microscopy system. Alternatively, or cumulatively, the connection device may also serve to establish an electrical connection between the apparatus and the microscopy system, the electrical connection being able to facilitate an energy supply of the apparatus and/or a signal transfer, for example a data transfer, from the apparatus to the microscopy system.

According to an aspect of the disclosure, the apparatus, in particular the apparatus-side connection device, has at least one element, or forms this element, for detection of a change in the connection state of the apparatus by the microscopy system.

The element for detection of the change in the connection state may include an element for detection of the connection state. The connection state may be a connected state, in which at least the aforementioned optical connection is established. Alternatively, the connection state may be a non-connected state, in which the aforementioned optical connection is not established. If the establishment or the cancellation of one of the states is detected, it is also possible to detect a change in the state.

The element for detection may be an optically detectable element, for example in the form of a QR code or a barcode. However, other optical detectable elements, for example optical markers, are naturally also conceivable. Additionally, the detectable element may be the apparatus-side light-guide element. In this case, the microscopy system may include an optical acquisition device for acquiring the optically detectable element.

Alternatively, the element for detection may be an electrically, mechanically, capacitively or inductively detectable element, or an element that is detectable in any other way. In such a case, the microscopy system must include a suitable acquisition device for detecting this element.

This microscopy system-side acquisition device and/or apparatus-side element for detection of the apparatus may be arranged and/or formed in such a way, for example, that the apparatus-side element of the apparatus is only arranged in the acquisition region of the optical acquisition device and/or detectable in a desired manner if the apparatus is in the connected state.

It is also conceivable that the element for detection includes an element for data transfer or signal transfer from the apparatus to the microscopy system, for example for unidirectional data transfer, with this transfer being able to be implemented in a wired or wireless fashion. A signal transfer for detection purposes may be a unidirectional signal transfer. Consequently, an element for data transfer or signal transfer may be formed as an element for unidirectional signal transfer as part of the element for detection.

It is also possible that the signal transfer for detection purposes is a one-time signal transfer and not a periodic or continuous signal transfer.

The microscopy system may include a corresponding receiver device for receiving the transferred signals/data. By way of example, in this case it is possible for the apparatus and/or the microscopy system, in particular the receiver device, to be arranged and/or formed such that the transfer is only possible, in particular only possible with a desired transfer quality, when the apparatus is in the connected state. An element for data transfer and/or signal transfer may be an RFID element, for example, which may be configured as a passive or active RFID element in particular.

Naturally, it is conceivable that alternatively configured elements for detection of a change in the connection state of the apparatus can be used by the microscopy system, these elements being configured such that a connected state and/or a non-connected state of the apparatus is detectable by the microscopy system.

The provided apparatus advantageously facilitates a reliable detection as to whether an external apparatus for OCT-based imaging is connected to the microscopy system-side connection device or whether the connected state of a connected apparatus is undone/released. This in turn advantageously facilitates the change-conditional adjustment of the mode of operation of the microscopy system, which will still be explained in more detail below. The change-conditional adjustment of the mode of operation in turn facilitates an adaptation of the mode of operation, performable in fully automated fashion in particular, to the connected apparatus, which firstly reduces the time for configuring this mode of operation and also increases the user-friendliness.

In a further exemplary embodiment, the apparatus, in particular the connection device, has at least one element, or forms this element, for identification of the apparatus by the microscopy system. The element for identification may form the element for detection or be part of said element. By way of example, the element for identification may encode an information item about an identifier of the apparatus or provide this information item in retrievable or readable fashion. There may be a bijection between the identifier and the apparatus.

It is possible for the microscopy system to include means for identifying a connected apparatus. These means may include, or be a part of, the means for detecting the connection state change.

It is further possible for these means and/or the apparatus to be arranged and/or designed or configured such that the apparatus is only identifiable in the connected state.

The presence of an element for identification of the apparatus by the microscopy system advantageously facilitates an identity-dependent adjustment of the mode of operation, which is likewise described in more detail below. This in turn facilitates an apparatus-specific adjustment of the mode of operation which simplifies a connection of different apparatuses and, in particular, reduces the time required to adapt the mode of operation of the microscopy system to different apparatuses and therefore likewise increases user-friendliness.

In a further exemplary embodiment, the apparatus, in particular the connection device of the apparatus, has at least one element, or forms this element, for readout of at least one apparatus-specific information item by the microscopy system.

By way of example, this element may be a memory device for the at least one apparatus-specific information item. Likewise, the element may have an interface for transferring signals/data, which encode the at least one apparatus-specific information item, from the apparatus to the microscopy system. This interface may facilitate—as explained above—a wireless or wired transfer. Additionally, the element for readout may encode the at least one apparatus-specific information item, for example in optical fashion, for example by a QR code or barcode. For example, an apparatus-specific information item may be an apparatus-specific parameter, exemplary parameters still being explained in more detail below.

It is possible for the microscopy system to include means for determining at least one apparatus-specific information item of a connected apparatus. By way of example, such means may include an interface for data transfer and/or signal transfer. These means may include, or be a part of, the means for detecting the connection state change. Additionally, these means may include, or be a part of, the identifying means. Thus, the same means or the same transmission route as for the readout of the at least one apparatus-specific information item and/or for identifying can be used for the detection of the connection state change.

It is further possible for these means and/or the apparatus to be arranged and/or designed or configured such that the at least one apparatus-specific information item can only be read in the connected state of the apparatus.

This advantageously renders an information item-dependent adjustment of the mode of operation of the microscopy system performable, which in turn facilitates an improved adaptation of the mode of operation to a connected apparatus. In particular, read information items can be used to adapt the mode of operation of the microscopy system to the specific connected apparatus. As a result of the apparatus providing the appropriate information items itself, the reliability of the adaptation is advantageously increased.

In a further exemplary embodiment, the apparatus-specific information item is an information item about a length of the apparatus-side light-guide element. Advantageously, this can adapt the operation of the OCT imaging unit, in particular the above-described OCT evaluation device, to the resultant length of the light-guide elements which connect the OCT radiation source to a radiation output coupling portion of the apparatus. By way of example, this information item can be used to adjust a length of a reference beam path of the OCT unit.

Alternatively, or cumulatively, the apparatus-specific information item is an information item about a diameter of the apparatus-side light-guide element. Further alternatively, or cumulatively, the information item may be an information item about a polarization characteristic of this light-guide element, more particularly an information item about whether the light-guide element is a polarization-maintaining light-guide element or a polarization-changing light-guide element. Further alternatively, or cumulatively, an apparatus-specific information item can be an information item about a distortion characteristic of the apparatus-side light-guide element. By way of example, such an information item may be an information item that the light-guide element is a non-distorting light-guide element or a distorting light-guide element.

The apparatus-specific information item may also be an information item about a transfer characteristic, in particular about a light transfer characteristic, of the apparatus. By way of example, such a characteristic may be a transmission factor which characterizes a transfer between an input coupling portion and an output coupling portion of the light-guide element.

An apparatus-specific information item may also be an information item about an admissible wavelength or an admissible wavelength range and/or about a maximum admissible power of the light or radiation to be transferred through the apparatus-side light-guide element.

By way of example, a characteristic of the radiation produced by the OCT radiation source can thus be adapted to the apparatus or its characteristics. By way of example, the power of the OCT radiation source can be adjusted such that the maximum admissible power of the radiation to be transferred through the apparatus-side light-guide element is not exceeded. Additionally, the wavelength for producing the radiation used by the OCT radiation source can be adjusted such that it corresponds to the admissible wavelength or is in the admissible wavelength range of the apparatus-side light-guide element.

Further, it is possible that the mode of operation of the OCT radiation source and/or the OCT evaluation device is adapted to the polarization characteristic of the apparatus-side light-guide element and/or to the distortion characteristic of the apparatus-side light-guide element, for example by virtue of performing or not performing a polarization compensation or by virtue of performing or not performing a distortion compensation.

Further, it is possible that a power of the radiation produced by the OCT radiation source is adjusted on the basis of transfer properties of the apparatus, in particular adjusted in such a way that a power radiated from an output coupling portion of the apparatus-side light-guide element to the examination object is as desired.

Further, the apparatus-specific information item may be an information item about an OCT imaging modality of the apparatus. By way of example, such an imaging modality may be an A-scan mode, a B-scan mode or else a vertical scan mode. Additionally, such an information item may indicate whether TD-OCT-based imaging or FD-OCT-based imaging or SS-OCT-based imaging is possible using the apparatus. In this case, a mode of operation of the OCT evaluation device, for example, may be adapted to the above-described OCT imaging modality of the apparatus. It is likewise possible to adjust the visualization of the OCT image signals, for example by way of a display device of the microscopy system, to the OCT imaging modality. For example, A-scan image information items may be represented differently than B-scan image information items.

The apparatus-specific information item may also be an information item about a field of application of the apparatus. The field of application may denote a surgical field or a spatial region. By way of example, the field of application may denote a region outside of the body or a region within the body. Additionally, the field of application may denote a spatial region of the human eye, for example a posterior eye portion or an anterior eye portion. In this case, a power of the OCT radiation source, for example, may be adapted to the field of application.

Additionally, a mode of operation of units of the microscopy system that differ from the OCT unit may be adjusted on the basis of the field of application, for example a mode of operation of an illumination device of the microscopy system which serves to illuminate the examination region. By way of example, the latter may be activated or deactivated depending on the field of application.

Additionally, the apparatus-specific information item may be an information item about an approval characteristic of the apparatus, for example an information item about a certification, for example a CE certification, or any other approval-relevant information item. In this case, the mode of operation of the microscopy system may be adapted to approval characteristics of the apparatus. By way of example, a transfer of OCT radiation to the connected apparatus may only be authorized if the apparatus has predetermined approval characteristics.

Additionally, the apparatus-specific information item may be an information item about a maximum admissible scanning rate of the OCT imaging using the apparatus. In this case, a mode of operation of the OCT evaluation device may be adapted to the maximum admissible scanning rate of the OCT imaging using the apparatus. By way of example, it is possible that apparatuses including active optical elements, for example mirror elements that are adjustable in terms of their position and/or orientation, may only be used with a comparatively lower scanning rate for imaging than apparatuses only including passive optical elements, for example lenses.

The above-described apparatus-specific information items advantageously facilitate an adaptation of the mode of operation of the microscopy system, in particular of the OCT radiation source and/or the OCT evaluation device, to characteristics of the connected apparatus.

Overall, this advantageously yields an improved operating quality on account of the information item-dependent adaptation of the mode of operation and also the aforementioned quick performance of this adaptation.

In a further exemplary embodiment, the apparatus has at least one element, or forms this element, for continuous and/or bidirectional signal transfer between the apparatus and the microscopy system.

This element may form the above-described element for signal transfer, which is part of the element for detection. Therefore, the signal transfer may be a wired or wireless data transfer. However, it is also possible that the element for continuous and/or bidirectional signal transfer differs from the element for signal transfer, which is part of the element for detection. Consequently, a signal transfer for detection may thus be implemented by way of a different element of the apparatus.

By way of example, it is possible for the apparatus to include at least one signal-receiving element, for example a controllable element, which receives control signals. In this case, the signals can be transferred from the microscopy system to the apparatus via the signal-receiving element. By way of example, a controllable element can be an optical element, for example a controllable lens and/or a mirror, the position and/or orientation of which is adjustable.

If the apparatus is an instrument, an operation of the instrument can be controlled by the signals transferred from the microscopy system to the instrument, the signals being transferred via the element for continuous and/or bidirectional signal transfer. By way of example, the instrument can be a cutting device with which, for example, openings can be introduced into the cornea, or a capsular bag can be cut open. In this case, a stop signal for terminating a cutting procedure, for example, may be transferred from the microscopy system to the apparatus if a continuation of the cutting procedure generates the risk of unwanted injury to body parts. The instrument may also be a suction apparatus, for example for aspirating a lens. In this case, too, a stop signal for terminating a sucking procedure, for example, may be transferred from the microscopy system to the apparatus if a continuation of the sucking procedure generates the risk of unwanted injury to body parts.

It is possible that signals between the apparatus and the microscopy system are transferred once, for example once after a connection state change, periodically or else continuously.

For example, it is possible that identifying and/or a readout of an apparatus-specific information item by the microscopy system or the signal transfer required to this end is implemented only once, in particular directly after the detection of a change of the connection state. In this case, the corresponding element must be configured only for a one-time signal transfer and not for a continuous signal transfer.

However, a periodic or continuous transfer facilitates, e.g., the dynamic control of an operation of controllable elements of an apparatus. Thus, it is also conceivable that the apparatus is a positioning device, for example a robot, or part thereof, which puts an end effector, e.g., an aspirator, a cutting device, an ablation device or a different end effector thereto, into desired positions and/or orientations during an operation, for the purposes of which actuators of the positioning device are operated accordingly. In this case, control signals can be transferred, for example continuously, from the microscopy system to the apparatus for the purposes of adjusting a target position and/or target orientation of the end effector. In particular, the positioning device can be a manually guided positioning device which a surgeon, for example, holds in their hand while an operation is performed.

It is also possible for the apparatus to include at least one signal-generating element, for example a sensor. By way of example, such a sensor may acquire an actual control quantity of an actuator. Additionally, a sensor may measure a temperature or a luminous intensity. A signal representing the acquired quantity may then be transferred continuously from the apparatus to the microscopy system. Then, operation of the microscopy system and/or the apparatus can be controlled on the basis of these signals, for example by an evaluation and control device of the microscopy system.

The continuous and/or bidirectional signal transfer advantageously facilitates an improved and simplified operation of an apparatus that is connected to the microscopy system.

Further, a microscopy system is provided, the latter including at least one OCT radiation source and at least one connection device for connecting an apparatus according to one of the embodiments explained in this disclosure to the microscopy system. The connection device of the microscopy system may also be referred to as microscopy system-side connection device. In respect of the design of the microscopy system, reference can be made to the explanations provided above. In a connected state, an optical connection is establishable between the OCT radiation source and the light-guide element of the apparatus in this case. This has already been explained above.

According to the disclosure, the microscopy system includes means for detecting a connection state change, a change-conditional adjustment of the mode of operation of the microscopy system being performable upon the detection of a connection state change. In particular, the microscopy system can be configured such that the change-conditional adjustment of the mode of operation is performed if a connection state change is detected. Exemplary adjustments of the mode of operation are explained in even greater detail below. Thus, expressed differently, the mode of operation of the microscopy system can be readjusted and, in particular, altered if a connection state change is detected, that is to say a change of an apparatus from a non-connected state to a connected state, or else from a connected state to a non-connected state.

As explained above, the microscopy system-side means for detecting a connection state change may also include means for detecting the connection state. By way of example, these are able to detect whether or not an external apparatus is optically and/or mechanically and/or electrically connected and/or connected in any other way to the connection device of the microscopy system. If there is a change in a connection state detected thus, it is possible to detect a connection state change.

The microscopy system-side means for detecting a connection state change may also include an element for data transfer or signal transfer from the apparatus to the microscopy system, with this transfer being able to be implemented in a wired or wireless fashion. Then, it is possible to detect a connection state change should such a transfer become possible in a non-connected state or should such a transfer be no longer possible, in particular no longer possible in a desired manner, in a connected state.

Further, the microscopy system may include at least one element for the above-described continuous and/or bidirectional signal transfer between the apparatus and the microscopy system. This element may form the aforementioned element for data transfer or signal transfer, which is part of the means for detection. However, it is also possible that the element for continuous and/or bidirectional signal transfer differs from the element for data transfer or signal transfer, which is part of the means for detection.

The microscopy system may include a control device which performs or controls the adjustment of the mode of operation. In this case, the control device may be embodied as or include a microcontroller or an integrated circuit.

By way of example, the change-conditional adjustment of the mode of operation can be implemented by the adjustment or the modification of at least one operational parameter of the microscopy system. The adjustment of the mode of operation being implemented in change-conditional fashion may mean that the provided adjustment is implemented if the connection state change was detected. By way of example, a trigger signal can be generated if a connection state change is detected. This trigger signal can trigger the change-conditional adjustment of the mode of operation, in particular if said trigger signal is received, for example, by the above-described control device. It is possible that the means for detecting the connection state change generates the trigger signal and transfers the latter to the control device.

Expressed differently, a change-conditional adjustment of the mode of operation of the microscopy system can be implemented in accordance with what is known as the plug-and-play principle. In this way, it is possible to adapt the mode of operation of the microscopy system, in particular, to a state that is given by the connection of a microscopy system-external apparatus for OCT imaging. It is also possible that there is an adaptation of the mode of operation to a state in which a previously connected apparatus is no longer connected. Advantageously, the provided microscopy system facilitates a quick and user-friendly adaptation of the mode of operation to the listed states.

In a further exemplary embodiment, the adjustment of the mode of operation is performable in partly automated fashion. This may mean that the adjustment of the mode of operation requires user interaction, for example a confirmation of the mode of operation to be set or of operational parameters to be set or a verification of a connected instrument, but not all of the steps required during the adjustment of the mode of operation need to be performed by the user.

Typically, the adjustment of the mode of operation when a connection state change is detected is performable in fully automated fashion, that is to say without interaction.

In particular, the microscopy system may be configured such that when the connection state change is detected, the change-conditional adjustment of the mode of operation of the microscopy system is performed, in particular in partly or fully automated fashion.

This advantageously saves time in relation to the user-based adjustment of the mode of operation that was explained at the outset, firstly reducing the time required to adapt the mode of operation and also increasing the user-friendliness.

In a further exemplary embodiment the microscopy system includes means for identifying a connected apparatus, an identity-dependent adjustment of the mode of operation being performable. In particular, the microscopy system can be configured such that the identity-dependent adjustment of the mode of operation is performed if the connected apparatus is identified.

By way of example, the identity-dependent adjustment may be an identity-specific adjustment of the mode of operation. In this case, different modes of operation may be assigned to different identities and hence also to different apparatuses. By way of example, a corresponding assignment may be stored in the form of a database, for example in a memory device of the microscopy system. Consequently, it is possible to determine the mode of operation assigned to a certain identity and then set said mode of operation. Naturally, it is also conceivable that the memory device is a microscopy system-external memory device, for example a memory device that is accessible via a network.

Advantageously, this results in the mode of operation being able to be adapted to apparatuses with different identities, that is to say, different apparatuses for OCT imaging, with however, likewise, the time required to set the adapted mode of operation being reduced and the user-friendliness being increased.

In a further exemplary embodiment, the microscopy system includes means for determining at least one apparatus-specific information item of a connected apparatus, an information item-dependent adjustment of the mode of operation being performable. In particular, the microscopy system can be configured such that the information item-dependent adjustment of the mode of operation is performed when an apparatus-specific information item is determined. By way of example, the means may facilitate the readout of an apparatus-specific information item from an apparatus-side memory device. However, it is also possible that the means facilitate the readout of apparatus-specific information items from an apparatus-external memory device, for example a memory device of the microscopy system or a memory device of a microscopy system-external server device. Additionally, the means for determining the at least one apparatus-specific information item may facilitate an optical acquisition, an inductive acquisition, a capacitive acquisition or another type of acquisition. Additionally, the means for determination may include elements for signal transfer or data transfer from the apparatus to the microscopy system, for example an element for the aforementioned unidirectional or bidirectional data transfer.

The adjustment of the mode of operation on the basis of an apparatus-specific information item advantageously facilitates a further improved adaptation of the mode of operation of the microscopy system to the connected apparatus. Exemplary apparatus-specific information items and the use thereof for adjusting the mode of operation have already been explained above.

In a further exemplary embodiment, an optical connection between the OCT radiation source and the light-guide element of a connected apparatus is established or separated by the change-conditional adjustment of the mode of operation. Additionally, an optical connection between the light-guide element and the OCT evaluation device may be established or separated. This has already been explained above. In particular, establishment and separation of said connection can be implemented by appropriately controlling an optical switching device. It is conceivable that the optical connection is established if a connection state change from a non-connected state to a connected state is/has been detected. If the optical connection between the OCT radiation source and the beam path of the microscopy system has been established, the aforementioned optical connection can be separated in the case of such a change.

It is further conceivable that the optical connection is separated if a connection state change from a connected state to a non-connected state is/has been detected. If the optical connection between the OCT radiation source and the beam path of the microscopy system has been separated, the aforementioned optical connection can be established in the case of such a change.

Advantageously, as a result, OCT imaging using a connected apparatus is facilitated or implemented as soon as the apparatus has been connected to the microscopy system. In particular, it is possible that this requires no further user interaction, for example a control of the aforementioned optical switching device, reducing the time required to activate the OCT imaging using the apparatus and increasing the user-friendliness.

Alternatively, or cumulatively, at least one characteristic of the radiation produced by the OCT radiation source, for example a power, a wavelength, a polarization state and/or a distortion state, is adjusted by the change-conditional adjustment of the mode of operation.

Advantageously, adjusting the mode of operation facilitates reliable and high-quality OCT imaging using the apparatus as a result, since the characteristics of the produced OCT radiation can be adapted to the characteristics of the apparatus.

Alternatively, or cumulatively, signal processing of the OCT signal is adjusted, this signal processing being implemented, in particular, by an evaluation device of the microscopy system. By way of example, this may include the adjustment of a scanning rate by the OCT evaluation device. Further, the adjustment may include the application of one or more signal filtering or image filtering methods and optionally the adjustment of desired filter characteristics. Additionally, the adjustment of the signal processing may include the application of one or more image processing methods and optionally the adjustment of desired processing characteristics. Signal filtering methods may be methods for compensating polarization and/or compensating distortion. By way of example, image processing methods may be segmentation methods or methods for feature identification. By way of example, adjusting desired filtering characteristics or image processing characteristics may be implemented by the adjustment of appropriate parameters of the corresponding methods. By way of example, should it be known that the connected apparatus facilitates OCT imaging with a low signal-to-noise ratio, the adjustment of the signal processing may include the application of appropriate noise filtering methods. Advantageously, this yields the generation of high-quality OCT image information items for an apparatus with a low signal-to-noise ratio.

Alternatively, or cumulatively, an operational state of at least one illumination device of the microscopy system may be adjusted by the change-conditional adjustment of the mode of operation. By way of example, should it be known that the field of application of the connected apparatus is a posterior eye portion, at least one light source of the microscopy system may be deactivated and, optionally, an external light source may be activated for illumination purposes. However, should it be known that the field of application is an anterior eye portion it is possible to activate the light source of the microscopy system. This therefore advantageously yields an improved adaptation of the mode of operation of the microscopy system, in particular functions of the microscopy system that go beyond the operation of the microscopy system-external, connected apparatus. This in turn increases the user-friendliness further.

Further alternatively, or cumulatively, the visualization of the OCT signal is adjusted by a visualization device of the microscopy system. Exemplary adjustments were explained above, that is to say, for example, the adjustment of the manner and/or the position of the display of the OCT image information items, adapted to the imaging modalities of the connected apparatus.

This further improves the user-friendliness of the microscopy system for a user, in particular, since facilitating a desired information visualization without user interaction for the adjustment thereof is achieved.

The operational state of the microscopy system adjusted by the change-conditional adjustment of the mode of operation may be assigned to the identity of the connected apparatus or to the above-described apparatus-specific information items. An operational state may also be referred to as a profile of the microscopy system, this profile therefore being able to be assigned to the identity or the apparatus-specific information items, for example in the form of an assignment known in advance which—as explained above—can be provided in the form of a database, for example. By way of example, an operational parameter of the OCT radiation source, that is to say for example a power of the produced OCT radiation, a wavelength, a polarization state or a distortion state, may be defined by such a profile. Expressed differently, the provided microscopy system thus facilitates an activation of an apparatus-specific profile.

In a further exemplary embodiment, a calibration is performable if a connection state change is detected, the change-conditional adjustment of the mode of operation then being performable on the basis of the result of the calibration. In particular, the microscopy system may be configured in such a way that the calibration is performed if the connection state change is detected and the change-conditional adjustment of the mode of operation is performed on the basis of the result of the calibration. By way of example, one of the above-described apparatus-specific information items may be determined by the calibration. Consequently, the calibration may refer to a method which serves to determine such an information item. Alternatively, at least one operational parameter of the microscopy system, in particular of the OCT unit, more particularly of the OCT radiation source and/or the OCT evaluation device, may be determined in such a way by the calibration that imaging with the desired quality is performable. Advantageously, this ensures that when an apparatus is connected, the information items required for operating the microscopy system with a desired quality of the OCT imaging by way of the external apparatus are determined quickly, without any user interaction being required, however.

In a further exemplary embodiment, a polarization optimization, a sweep signal search or a signal-to-noise ratio estimate is performed by the calibration. In this case, the specified methods are known to the person skilled in the art. Advantageously, this facilitates a quick and good adaptation of the mode of operation of the OCT unit of the microscopy system to unknown (optical) characteristics of a (newly) connected apparatus, which in turn ensures a desired high imaging quality of the OCT imaging using the connected apparatus.

A method is moreover provided for operating a microscopy system which is configured according to one of the embodiments described in this disclosure. In this case, a change-conditional adjustment of the mode of operation of the microscopy system is performed when a connection state change is detected. In this case, the method is performed with or by the aforementioned microscopy system. Consequently, the microscopy system is configured in such a way that a method according to one of the exemplary embodiments described in this disclosure can be performed by the microscopy system.

It is possible for the microscopy system to include means for monitoring the connection state. These are able to determine the connection state, for example continuously, periodically or at selected times. If the connection state has changed in comparison with the last-determined state, it is possible to detect a connection state change. However, in the case of a connection state change, it is self-evidently conceivable that a corresponding change signal can be generated, and consequently can be detected, even without monitoring.

The method may further include the connection of an apparatus, or the decoupling of the apparatus, from the microscopy system. Technical advantages of the change-conditional adjustment of the mode of operation were already explained above and are also obtained by the provided method.

In a further exemplary embodiment, a connected apparatus is identified and/or at least one apparatus-specific information item of the connected apparatus is determined, with an identity-dependent and/or information item-dependent adjustment of the mode of operation of the microscopy system being performed. This and corresponding technical advantages have been explained above.

In a further exemplary embodiment, at least one apparatus-specific information item is determined on the basis of the identity of the apparatus. By way of example, the apparatus-specific information item may be determined on the basis of an assignment, for example in the form of a database, with which the at least one apparatus-specific information item is assigned to an identity of an apparatus. This and corresponding technical advantages have likewise been explained above.

In an alternative exemplary embodiment, the at least one apparatus-specific information item is read from an apparatus-side element for readout of the at least one apparatus-specific information item. This and corresponding advantages have likewise been explained above.

Further described is a system including a microscopy system according to one of the exemplary embodiments described in this disclosure and an apparatus for OCT-based imaging according to one of the exemplary embodiments described in this disclosure, the apparatus being able to be connected to the microscopy system.

### BRIEF DESCRIPTION OF THE DRAWINGS

The disclosure will now be described with reference to the drawings wherein:

FIG. 1 shows a schematic illustration of an apparatus for OCT imaging according to an exemplary embodiment of the disclosure,

FIG. 2 shows a schematic illustration of a microscopy system according to an exemplary embodiment of the disclosure,

FIG. 3A shows a schematic illustration of an apparatus-side connection device according to a first exemplary embodiment of the disclosure,

FIG. 3B shows a schematic illustration of an apparatus-side connection device in a further exemplary embodiment of the disclosure,

FIG. 3C shows a schematic illustration of an apparatus-side connection device in a further exemplary embodiment of the disclosure,

FIG. 4 shows a schematic illustration of a system including a microscopy system and an apparatus for OCT imaging,

FIG. 5 shows a flowchart of a method according to an exemplary embodiment of the disclosure,

FIG. 6 shows a flowchart of a method according to a further exemplary embodiment of the disclosure, and

FIG. 7 shows a flowchart of a method according to a further exemplary embodiment of the disclosure.

### DESCRIPTION OF EXEMPLARY EMBODIMENTS

Identical reference signs hereinafter denote elements having identical or similar technical features.

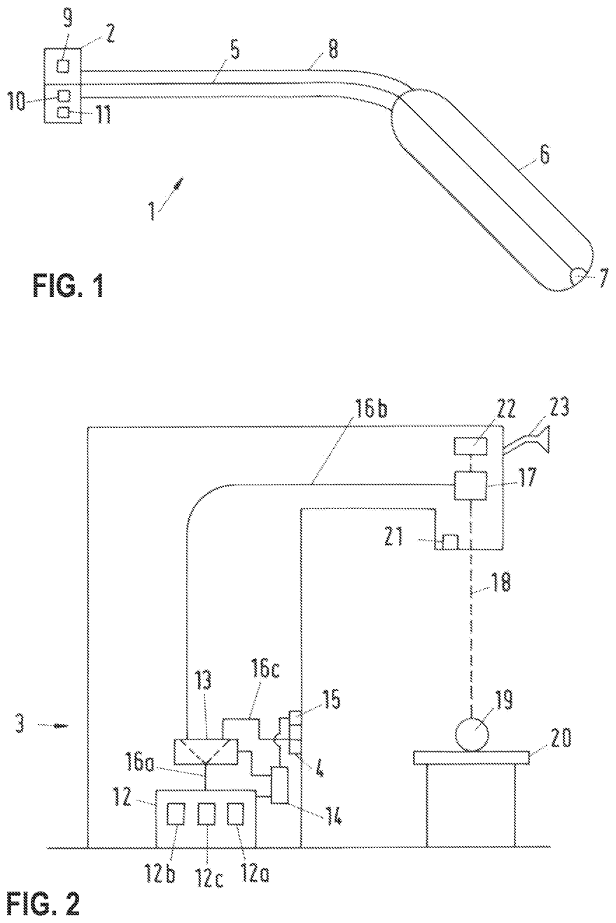

FIG. 1 shows a schematic illustration of an apparatus 1 for OCT-based imaging. The apparatus includes a connection device 2 for connecting the apparatus 1 to a microscopy system 3 (see FIG. 2), in particular to a microscopy system-side connection device 4. Further, the apparatus 1 includes a light-guide element 5, which is also referred to as apparatus-side light-guide element 5. What is further shown is that the apparatus 1 has an actuation body 6 for manual actuation/handling of the apparatus 1. Shown further is an output coupling portion 7 at one end of the actuation body 6, the output coupling portion being used to output couple OCT radiation from the light-guide element 5 into the surround, in particular into an examination region. Naturally, this output coupling portion 7 may also serve to input couple light that was reflected by the examination region into the light-guide element 5. In this case, the light-guide element 5 extends from the connection device 2 to the aforementioned output coupling portion 7. Shown further is a protective sleeve 8 for the light-guide element 5, through which the light-guide element 5 is guided from the connection device 2 to the actuation body 6.

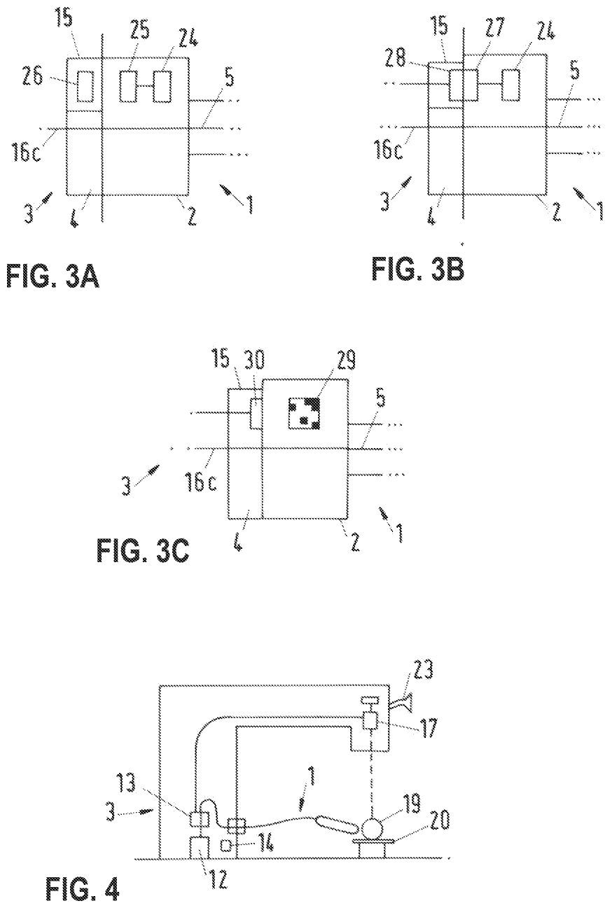

The apparatus 1, in particular the connection device 2, has an element 9 for detection of a change in the connection state of the apparatus 1 by the microscopy system 2. Exemplary forms of such an element 9 are still explained in more detail below, in particular in relation to the embodiments in FIGS. 3A, 3B, and 3C.

In this case, this element 9 is arranged and/or formed such that microscopy system-side means for detection are able to detect this element 9 if the apparatus 1 is connected to the microscopy system 3 by way of the connection device 2 (see the state illustrated in FIG. 4). By way of example, an acquisition region may be assigned to the microscopy system-side means for detection, the element 9 of the apparatus 1 only being located in the acquisition region of the means and/or the means for detection only being able to detect the element 9 with a predetermined detection reliability when said element is connected to the microscopy system 1.

Shown further is that the apparatus 1, in particular the connection device 2, has an element 10 for identification of the apparatus 1 by the microscopy system 3. This element 10 for identification may be in the form of a barcode or an RFID element, for example, which is acquirable/readable by microscopy system-side identification means. It is possible that the microscopy system-side identification means and/or the element 10 for identification are arranged and/or formed such that identifying is only possible and/or only possible with a predetermined reliability if the apparatus 1 is connected to the microscopy system 2, that is to say if the apparatus is in the connected state.

Shown further is that the apparatus 1 has an element 11 for readout of at least one apparatus-specific information item by the microscopy system 3. By way of example, this element 11 may likewise be in the form of an RFID element. Alternatively, this element 11 may include a memory device and a device for information transfer, for example in the form of a signal transfer and/or data transfer. This element may be configured for unidirectional data transfer or for bidirectional data transfer. The microscopy system 3 may include means for reading this information. It is conceivable that the reading means and/or the element 11 for readout are arranged and/or formed such that the apparatus-specific information item can only be read, in particular only be read with a predetermined reliability/quality, by the microscopy system 3 in the connected state of the apparatus 1.

FIG. 3 illustrates that the element 9 for detection of the change, the element 10 for identification and element 11 for readout are formed as separate elements. However, this is not mandatory. Thus, it is conceivable that the element 9 for detection includes or is formed by the element 10 for identification and/or the element 11 for readout. It is further possible that the element 10 for identification includes or is formed by the element 11 for readout.

Further, it is possible that the detection of the change in the connection state, the identification or the readout is implemented by the same means of the microscopy system 3. By way of example, if the microscopy system 3 includes a sensor for detecting the change of the connection state or for detecting the connection state, this sensor may also be used for the identification of the apparatus 1 by the microscopy system 3 and/or for the readout of at least one apparatus-specific information item by the microscopy system 3. By way of example, such a sensor may be an optical sensor.

Alternatively, it is conceivable that the microscopy system-side reading means includes a receiver device for signals/data transferred by the apparatus 1 in wireless or wired fashion. These means may also be formed for unidirectional or bidirectional signal transfer.

Then, this means can likewise be used to identify the apparatus 1 and/or to detect the change in the connection state. In particular, signals/data transferred from the apparatus 1 to the microscopy system 3 may be used to detect the change in the connection state and to identify and/or read out the specific information item. If the means is configured for bidirectional signal transfer, it is possible, e.g., to transfer control signals for operating a controllable element of the apparatus 1 to the latter.

FIG. 2 shows a schematic illustration of a microscopy system 3. This microscopy system 3 includes an OCT unit 12, this OCT unit 12 including an OCT radiation source 12a, an OCT reference beam path 12b and an OCT evaluation device 12c, which may also be referred to as an OCT detector. Further, the microscopy system 3 illustrated in FIG. 2 includes an optical switching device 13, which may be configured, but is not necessarily configured, as a changeover switching device. Shown further is that the microscopy system 3 includes an evaluation and control device 14 which may be in the form of, or include, a microcontroller or integrated circuit, for example.

Shown further is a microscopy system-side connection device 4 for the connection of an apparatus 1 (see FIG. 1), in particular for the connection of the apparatus-side connection device 2. Likewise shown is a device 15 for detection of a change in the connection state of the apparatus 1 by the microscopy system 3. In this case, the device 15 can form or be part of the above-described means for detection of the change of the connection state. The device 15 may likewise serve for identification of a connected apparatus 1. Likewise, the device 15 may serve for readout of an apparatus-specific information item by the microscopy system 3. What is shown is that this device 15, the optical switching device 13 and the OCT unit 12 are data-connected to the control and evaluation device 14, for example by way of suitable data lines and/or signal lines, in a further example by way of a bus system.

Shown further is that the OCT unit 12 is connected to the optical switching device 13 by way of a first microscopy system-side light-guide element 16a. Shown further is that the optical switching device 13 is connected via a second microscopy system-side light-guide element 16b to a beam splitter 17 of the microscopy system 3, with which OCT radiation can be input coupled into a schematically illustrated beam path 18 of the microscopy system 3 and can be output coupled from this beam path 18. A third microscopy system-side light-guide element 16c is likewise shown, the optical switching device 13 being connected to the microscopy system-side connection device 4 therewith. Shown further is a patient 19 on an operating table 20, the patient 19 likewise being arranged in the beam path of the microscopy system 3.

In a first switching state of the optical switching device 13, the OCT unit 12 is connected to the beam splitter 17 via the first and the second light-guide element 16a, 16b. In a second switching state, the OCT unit 12 is connected to the microscopy system-side connection device 4 via the first and the third light-guide element 16a, 16c. In this case, the control and evaluation device 14 can adjust the switching states of the optical switching device 13.

If an apparatus 1 is connected to the microscopy system-side connection device 4, the third microscopy system-side light-guide element 16c is optically connected to the apparatus-side light-guide element 5. Consequently, the OCT unit 12 is also connected to the output coupling portion 7 of the apparatus 1, illustrated in FIG. 1, via the first microscopy system-side light-guide element 16a, the third microscopy system-side light-guide element 16c and the apparatus-side light-guide element 5. This facilitates OCT imaging by way of the apparatus 1 illustrated in FIG. 1, the OCT unit 12 of the microscopy system 3 being used for the imaging.

FIG. 2 further shows an illumination device 21 of the microscopy system 3, which can illuminate an optical acquisition region of said microscopy system 3. Likewise shown is an imaging device 22 for generating non-OCT-based image information items of the examination region of the microscopy system 3, for example of the patient 19. Likewise shown is an eyepiece 23, which may be in the form of a stereo eyepiece, for example, and which is coupled to the beam path 18. In this case, the shown control and evaluation device 14 can control an operation of the illumination device and of the imaging device 22.

FIG. 3A shows a schematic illustration of a connection device 2 of an apparatus 1 for OCT imaging and a portion of the microscopy system 3. Shown here is a connected state, in which the apparatus-side connection device 2 is connected, in particular mechanically connected, to the microscopy system-side connection device 4, in such a way that an optical connection is established between the third microscopy system-side light-guide element 16c, illustrated in FIG. 2, and the apparatus-side light-guide element 5. Shown further is a memory device 24 and a transmitter device 25 of the apparatus 1, in particular of the connection device 2. In this case, the memory device 24 can store information items about an identity of the apparatus 1 and/or apparatus-specific information items such as, for example, information items about a length and/or a diameter of the apparatus-side light-guide element 5 and optionally further characteristics specified in this disclosure.

The microscopy system 3, in particular the device 15, includes a receiver device 26 for receiving signals/data which are transmitted from the apparatus-side transmitter device 25 to the receiver device 26. Consequently, the above-described information items can be transmitted from the apparatus 1 to the microscopy system 3. Further, these information items can then be transmitted from the device 15 to the control and evaluation 14, which can then adjust a mode of operation of the microscopy system 3 in a manner adapted to the above-described information items. It is possible for the transmitter device 25 to be in the form of a readable RFID transponder. Further, the receiver device 26 may be an RFID reader for readout. The receiver device 26 can be part of a transmitter and receiver device which facilitates a bidirectional signal transfer, that is to say a signal transfer from the apparatus 1 to the microscopy system 3, and vice versa. However, it is also possible for the receiver device 26 to be configured for unidirectional signal transfer only, that is to say from the apparatus 1 to the microscopy system 3.

In the embodiment illustrated in FIG. 3A, the receiver device 26 may be arranged or configured such that the transmitter device 25 is only in the reception region of this receiver device 26 if the apparatus is connected to the microscopy system 3 in the desired way.

FIG. 3B shows a schematic illustration of an apparatus-side connection device 2 according to a further exemplary embodiment. This connection device 2 also includes a memory device 24 for storing the above-described information items. Shown further is that the connection device 2 includes an interface 27 for wired data transfer. The microscopy system-side device 15 likewise includes an interface 28 for wired data transfer. If the apparatus-side connection device 2 is connected to the microscopy system-side connection device 4, an optical connection is firstly established between the light-guide elements 16c, 5, as is a data connection between the microscopy system 3 and the apparatus 1, in particular via the interfaces 27, 28. This facilitates a readout of the information items stored in the memory device 24, which can then be transferred to the control and evaluation device 14.

In the exemplary embodiment illustrated in FIG. 3B, it is possible to detect a change in the connection state if a data transfer and/or signal transfer via the interfaces 27, 28 which was not possible previously is rendered possible, or vice versa.

FIG. 3C shows a schematic illustration of a connection device 2 according to the exemplary embodiment of disclosure of an apparatus 1 for OCT-based imaging according to a further exemplary embodiment. In this case, the connection device 2 includes a marker 29 in the form of a QR code. The device 15 of the microscopy system includes an image acquisition device 30 for optically acquiring the marker 29. In this case, the acquisition region of this optical acquisition device 30 can be arranged and/or formed such that the QR code can only be acquired if the connection device 2 is connected to the connection device 4 of the microscopy system 3 in the desired manner and hence an optical connection has been established between the microscope-side and apparatus-side light-guide elements 16c, 5.

FIG. 4 shows a schematic illustration of a microscopy system with an apparatus 1 for OCT-based imaging connected thereto. In this state, the switching state of the optical switching device 13 can be adjusted by the control and evaluation device 14 in such a way that the OCT unit 12 is connected to the apparatus 1 but not to the beam splitter 17. If this connected state is undone and the corresponding connection state change is detected, the control and evaluation device 14 can control the optical switching device 13 such that a switching state in which the OCT unit 12 is connected to the beam splitter 17 but no longer connected to the apparatus 1 is set.

The control and evaluation device 14 can adjust the mode of operation of the microscopy system 3 if a connection state change is detected.

What is not shown is that the microscopy system 3 may include a memory device for storing apparatus-specific information items, typically of a plurality of apparatuses 1. By way of this memory device, it is possible, for example, to store an assignment of identities of these apparatuses 1 to the corresponding apparatus-specific information items. Should an identity of a connected apparatus 1 subsequently be determined, apparatus-specific information items may then be retrieved from this memory device, it then being possible to perform an information item-dependent adjustment of the mode of operation. It is conceivable that this memory device is a memory device of the microscopy system 3, but also a microscope-external memory device, for example a memory device that is callable via a network.

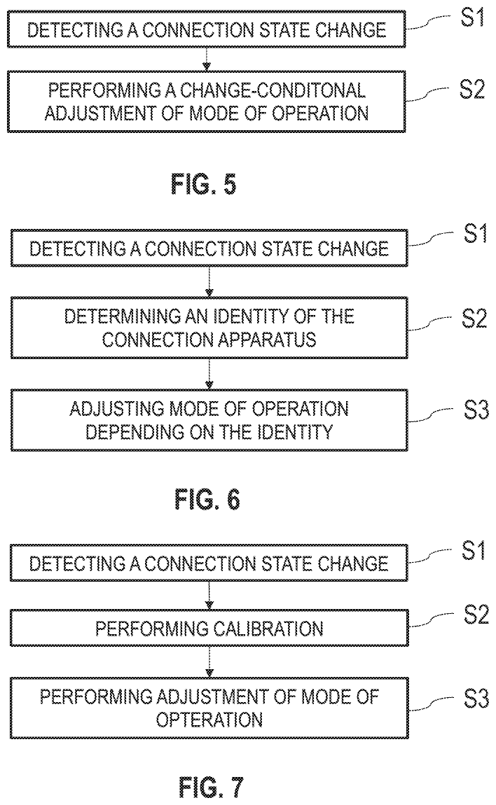

FIG. 5 shows a schematic flowchart of a method according to the disclosure for operating a microscopy system 3 (see FIG. 2) according to a first exemplary embodiment. In this case, a connection state change—as explained above—is detected in a first step S1. Then, a change-conditional adjustment of the mode of operation of the microscopy system 3 is performed in a second step S2. In particular, the optical switching device 13 of the microscopy system 3 can be controlled, or the switching state thereof can be adjusted, in connection state-dependent fashion.

FIG. 6 shows a schematic flowchart of a method according to a further exemplary embodiment of the disclosure. In this case, a connection state change is detected in a first step S1. If a connected state of an apparatus 1 (see FIG. 1) is detected, an identity of the connected apparatus 1 and/or an apparatus-specific information item can be determined in a second step S2. Then, an identity-dependent and/or information item-dependent adjustment of the mode of operation of the microscopy system 3 can be implemented in a third step S3.

FIG. 7 shows a schematic flowchart of a method according to a further exemplary embodiment of the disclosure. In this case, a connection state change can be detected in a first step S1. Then, a calibration, for example including a polarization optimization, a sweep signal search or a signal-to-noise ratio estimate, can be performed in a second step S2. Then, an adjustment of the mode of operation of the microscopy system 3 can be performed on the basis of the result of the calibration in a third step S3.

It is understood that the foregoing description is that of the exemplary embodiments of the disclosure and that various changes and modifications may be made thereto without departing from the spirit and scope of the disclosure as defined in the appended claims.

### LIST OF REFERENCE NUMERALS

1 Apparatus
2 Connection device
3 Microscopy system
4 Microscopy system-side connection device
5 Apparatus-side light-guide element
6 Actuation body
7 Output coupling portion
8 Protective sleeve
9 Element for detection
10 Element for identifying
11 Element for readout
12 OCT unit
12a OCT radiation source
12b OCT reference beam path
12c OCT evaluation device
13 Optical switching device
14 Control and evaluation device
15 Device for detection
16a, 16b, 16c Microscopy system-side light-guide element
17 Beam splitter
18 Beam path
19 Patient
20 Operating table
21 Illumination device
22 Imaging device
23 Eyepiece
24 Memory device
25 Transmitter device
26 Receiver device
27 Interface
28 Interface
29 Marker
30 Image acquisition device
S1 First step
S2 Second step
S3 Third step

What is claimed is:

1. An apparatus for OCT-based imaging, the apparatus comprising:
at least one light-guide element;
at least one connection device for connecting the apparatus to a microscopy system with an OCT radiation source;
an optical connection being configured to be established between the OCT radiation source and the at least one light-guide element in a connected state; and
the apparatus having or forming at least one element for detecting a change in the connection state of the apparatus by the microscopy system.

2. The apparatus according to claim 1, wherein the apparatus has or forms at least one element for identification of the apparatus by the microscopy system.

3. The apparatus according to claim 1, wherein the apparatus has or forms at least one element for readout of at least one apparatus-specific information item by the microscopy system.

4. The apparatus according to claim 3, wherein the at least one apparatus-specific information item is an information item about at least one of:
at least one of a length and a diameter of the at least one light-guide element,
a polarization characteristic of the at least one light-guide element,
a distortion characteristic of the at least one light-guide element,
a transfer characteristic of the apparatus,
a maximum admissible power of radiation to be transferred through the at least one light-guide element,
an admissible wavelength or an admissible wavelength range of the radiation to be transferred through the at least one light-guide element,
an OCT imaging modality of the apparatus,
a field of application of the apparatus,
an approval characteristic of the apparatus, and
a maximum admissible scanning rate of the OCT imaging using the apparatus.

5. The apparatus according to claim 1, wherein the apparatus has or forms at least one of an element for continuous and bidirectional data transfer between the apparatus and the microscopy system.

6. A microscopy system, comprising:
at least one OCT radiation source and at least one connection device for connecting an apparatus according to claim 1 to the microscopy system; and
the optical connection being configured to be established between the OCT radiation source and the at least one light-guide element of the apparatus in a connected state; and
means for detecting a connection state change, and
wherein a change-conditional adjustment of the mode of operation of the microscopy system is performable upon a detection of the connection state change.

7. The microscopy system according to claim 6, wherein the change-conditional adjustment is performable in a partly or fully automatic fashion.

8. The microscopy system according to claim 6, further comprising:
means for identifying a connected apparatus, and
wherein the microscopy system is configured to perform an identity-dependent adjustment of the mode of operation.

9. The microscopy system according to claim 6, further comprising:
means for determining at least one apparatus-specific information item of a connected apparatus, and
wherein the microscopy system is configured to perform an information item-dependent adjustment of the mode of operation.

10. The microscopy system according to claim 6, wherein microscopy system is configured to at least one of:
establish or separate the optical connection between the OCT radiation source and the at least one light-guide element of a connected apparatus,
adjust at least one characteristic of radiation generated by the OCT radiation source,
adjust signal processing of an OCT signal,
adjust an operational state of at least one illumination device of the microscopy system, and
adjust a visualization of the OCT signal by a visualization device of the microscopy system as a result of the change-conditional adjustment of the mode of operation.

11. The microscopy system according to claim 6, wherein the microscopy system is configured to:
perform a calibration when a connection state change is detected,
perform the change-conditional adjustment of the mode of operation based on a result of the calibration.

12. The microscopy system according to claim 11, wherein the microscopy system is configured to:
perform at least one of a polarization optimization, a sweep signal search, and a signal-to-noise ratio estimate by the calibration.

13. A method for operating the microscopy system according to claim 6, the method comprising:
performing the change-conditional adjustment of the mode of operation of the microscopy system when a connection state change is detected.

14. The method according to claim 13, further comprising at least one of:
identifying a connected apparatus;
determining at least one apparatus-specific information item of the connected apparatus; and
performing at least one of an identity-dependent and information item-dependent adjustment of the mode of operation of the microscopy system.

15. The method according to claim 14, further comprising:
determining at least one apparatus-specific information item based on the identity of the apparatus.

16. The method according to claim 14, further comprising:
reading the at least one apparatus-specific information item from an apparatus-side element for readout of the at least one apparatus-specific information item.
